(19) United States
(12) Patent Application Publication
(10) Pub. No.: US 2016/0281117 A1
(43) Pub. Date: Sep. 29, 2016

(54) PROCESSES FOR CONSUMING ACETIC ACID DURING FERMENTATION OF CELLULOSIC SUGARS, AND PRODUCTS PRODUCED THEREFROM

(71) Applicant: API Intellectual Property Holdings, LLC, Atlanta, GA (US)

(72) Inventors: Theodora RETSINA, Atlanta, GA (US); David GLASSNER, Littleton, CO (US); Ryan O'CONNOR, Minnetrista, MN (US)

(21) Appl. No.: 15/082,826

(22) Filed: Mar. 28, 2016

Related U.S. Application Data
(60) Provisional application No. 62/139,003, filed on Mar. 27, 2015.
(60) Provisional application No. 62/139,005, filed on Mar. 27, 2015.

(51) Int. Cl. C12P 7/64; C12P 7/10

(52) U.S. Cl. CPC C12P 7/64; C12P 7/10; C12P 2203/00

(57) ABSTRACT

This invention provides a way to deal with acetic acid derived from biomass, for fermentation of cellulosic sugars. In some variations, a process for producing ethanol from lignocellulosic biomass comprises: extracting hemicelluloses and acetic acid from lignocellulosic biomass; hydrolyzing the hemicelluloses, using an acid catalyst or enzymes, to generate hemicellulose monomers and more acetic acid; fermenting acetic acid to lipids using a suitable lipid-producing microorganism, thereby reducing acetic acid concentration; fermenting hemicellulose monomers to ethanol using a suitable ethanol-producing microorganism; and recovering the ethanol. The co-fermentation of acetic acid and sugars may be carried out in a single fermentor or in separate fermentors. The invention may be applied to fermentation products other than ethanol. In some embodiments, the fermentation product can act as an extraction solvent to extract lipids from the lipid-producing microorganism, such as a lipid-producing yeast.

### PRIORITY DATA

This patent application is a non-provisional application claiming priority to U.S. Provisional Patent App. No. 62/139,003 filed Mar. 27, 2016 and to U.S. Provisional Patent App. No. 62/139,005 filed Mar. 27, 2016, each of which is hereby incorporated by reference herein.

### FIELD

The present invention generally relates to biorefining processes for converting biomass into fermentable sugars.

### BACKGROUND

Lignocellulosic biomass is the most abundant renewable material on the planet and has long been recognized as a potential feedstock for producing chemicals, fuels, and materials. Lignocellulosic biomass normally comprises primarily cellulose, hemicellulose, and lignin. Cellulose and hemicellulose are natural polymers of sugars, and lignin is an aromatic/aliphatic hydrocarbon polymer reinforcing the entire biomass network.

When it is desired to produce fermentable sugars biomass, a significant challenge is caused by the presence of acetyl groups in the biomass. The acetyl groups are released into solution during initial fractionation or extraction, and during hydrolysis of oligomers (hemicelluloses). The acetic acid is a known, potent inhibitor of many industrial fermentation processes, such as sugar fermentation to ethanol.

Improved processes to manage and reduce acetic acid are needed commercially for biorefineries producing fermentable sugars.

### SUMMARY

The present invention addresses the aforementioned needs in the art.

In some variations, the invention provides a process for producing ethanol from lignocellulosic biomass, the process comprising:

(a) extracting hemicelluloses and a first amount of acetic acid from a feedstock comprising lignocellulosic biomass;

(b) hydrolyzing the hemicelluloses, using an acid catalyst or enzymes, to generate hemicellulose monomers and a second amount of acetic acid;

(c) fermenting at least a portion of the acetic acid to lipids using a suitable lipid-producing microorganism, thereby reducing acetic acid concentration;

(d) optionally removing at least a portion of the lipids, during or following step (c);

(e) fermenting at least a portion of the hemicellulose monomers to ethanol using a suitable ethanol-producing microorganism; and

(f) recovering the ethanol.

In some variations, the invention provides a process for producing ethanol from lignocellulosic biomass, the process comprising:

(a) extracting hemicelluloses and a first amount of acetic acid from a feedstock comprising lignocellulosic biomass;

(b) hydrolyzing the hemicelluloses, using an acid catalyst or enzymes, to generate hemicellulose monomers and a second amount of acetic acid;

(c) in a single fermentor, fermenting at least a portion of the acetic acid to lipids using a suitable lipid-producing microorganism, thereby reducing acetic acid concentration, while also fermenting at least a portion of the hemicellulose monomers to ethanol using a suitable ethanol-producing microorganism that is different than the lipid-producing microorganism;

(d) optionally removing at least a portion of the lipids, during or following step (c); and

(e) recovering the ethanol.

In some variations, the invention provides a process for producing ethanol from lignocellulosic biomass, the process comprising:

(a) extracting hemicelluloses and a first amount of acetic acid from a feedstock comprising lignocellulosic biomass;

(b) hydrolyzing the hemicelluloses, using an acid catalyst or enzymes, to generate hemicellulose monomers and a second amount of acetic acid;

(c) in a single fermentor, fermenting at least a portion of the acetic acid to lipids using a suitable lipid-producing microorganism, thereby reducing acetic acid concentration, while also fermenting at least a portion of the hemicellulose monomers to ethanol using the lipid-producing microorganism that is also capable of producing ethanol from the hemicellulose monomers;

(d) optionally removing at least a portion of the lipids, during or following step (c); and

(e) recovering the ethanol.

In some variations, the invention provides a process for producing ethanol from lignocellulosic biomass, the process comprising:

(a) extracting hemicelluloses and a first amount of acetic acid from a feedstock comprising lignocellulosic biomass;

(b) hydrolyzing the hemicelluloses, using an acid catalyst or enzymes, to generate hemicellulose monomers and a second amount of acetic acid;

(c) fermenting at least a portion of the acetic acid to CO₂ and/or cell mass using a suitable acetic acid-consuming microorganism, thereby reducing acetic acid concentration;

(d) optionally removing at least a portion of the CO₂ and/or cell mass, during or following step (c);

(e) fermenting at least a portion of the hemicellulose monomers to ethanol using a suitable ethanol-producing microorganism; and

(f) recovering the ethanol.

In some variations, the invention provides a process for producing ethanol from lignocellulosic biomass, the process comprising:

(a) extracting hemicelluloses and a first amount of acetic acid from a feedstock comprising lignocellulosic biomass;

(b) hydrolyzing the hemicelluloses, using an acid catalyst or enzymes, to generate hemicellulose monomers and a second amount of acetic acid;

(c) in a single fermentor, fermenting at least a portion of the acetic acid to CO₂ and/or cell mass using a suitable acetic acid-consuming microorganism, thereby reducing acetic acid concentration, while also fermenting at least a portion of the hemicellulose monomers to ethanol using a suitable ethanol-producing microorganism that is different than the acetic acid-consuming microorganism;

(d) optionally removing at least a portion of the CO₂ and/or cell mass, during or following step (c); and

(e) recovering the ethanol.

In some variations, the invention provides a process for producing ethanol from lignocellulosic biomass, the process comprising:

(a) extracting hemicelluloses and a first amount of acetic acid from a feedstock comprising lignocellulosic biomass;

(b) hydrolyzing the hemicelluloses, using an acid catalyst or enzymes, to generate hemicellulose monomers and a second amount of acetic acid;

(c) in a single fermentor, fermenting at least a portion of the acetic acid to CO₂ and/or cell mass using a suitable acetic acid-consuming microorganism, thereby reducing acetic acid concentration, while also fermenting at least a portion of the hemicellulose monomers to ethanol using the acetic acid-consuming microorganism that is also capable of producing ethanol from the hemicellulose monomers;

(d) optionally removing at least a portion of the CO₂ and/or cell mass, during or following step (c); and

(e) recovering the ethanol.

In some variations, the invention provides a process for producing lipids from lignocellulosic biomass, the process comprising:

(a) extracting hemicelluloses and a first amount of acetic acid from a feedstock comprising lignocellulosic biomass;

(b) hydrolyzing the hemicelluloses, using an acid catalyst or enzymes, to generate hemicellulose monomers and a second amount of acetic acid;

(c) fermenting at least a portion of the acetic acid to lipids using a suitable lipid-producing microorganism, thereby reducing acetic acid concentration;

(d) optionally removing at least a portion of the lipids, during or following step (c);

(e) fermenting at least a portion of the hemicellulose monomers to a fermentation product using a suitable microorganism; and

(f) recovering the lipids.

In some variations, the invention provides a process for producing lipids from lignocellulosic biomass, the process comprising:

(a) extracting hemicelluloses and a first amount of acetic acid from a feedstock comprising lignocellulosic biomass;

(b) hydrolyzing the hemicelluloses, using an acid catalyst or enzymes, to generate hemicellulose monomers and a second amount of acetic acid;

(c) in a single fermentor, fermenting at least a portion of the acetic acid to lipids using a suitable lipid-producing microorganism, thereby reducing acetic acid concentration, while also fermenting at least a portion of the hemicellulose monomers to a fermentation product using a suitable microorganism that is different than the lipid-producing microorganism;

(d) optionally removing at least a portion of the lipids, during or following step (c); and

(e) recovering the lipids.

In some variations, the invention provides a process for producing lipids from lignocellulosic biomass, the process comprising:

(a) extracting hemicelluloses and a first amount of acetic acid from a feedstock comprising lignocellulosic biomass;

(b) hydrolyzing the hemicelluloses, using an acid catalyst or enzymes, to generate hemicellulose monomers and a second amount of acetic acid;

(c) in a single fermentor, fermenting at least a portion of the acetic acid to lipids using a suitable lipid-producing microorganism, thereby reducing acetic acid concentration, while also fermenting at least a portion of the hemicellulose monomers to a fermentation product using the lipid-producing microorganism that is also capable of producing the fermentation product from the hemicellulose monomers;

(d) optionally removing at least a portion of the lipids, during or following step (c); and

(e) recovering the lipids.

In some embodiments, the fermentation product is capable of extracting the lipids from the lipid-producing microorganism. Optionally, the process of the invention further comprises extracting the lipids from the lipid-producing microorganism using an extraction solvent comprising the fermentation product. For example, the fermentation product may be selected from the group consisting of C₄ or higher alcohols, C₄ or higher fatty acids, C₄ or higher alkanes, and combinations thereof. In some embodiments, the extraction solvent comprises ethanol. In some embodiments, the extraction solvent further comprises lignin or a lignin derivative.

In some embodiments, step (a) utilizes steam and/or hot water, optionally with an extraction catalyst. In some embodiments, step (a) utilizes an extraction catalyst, a solvent for lignin, and water. In some embodiments, step (b) utilizes a sulfur-containing acid catalyst. Step (b) may utilize an acid catalyst derived from step (a).

In some embodiments of the invention, at least one of the microorganisms is a genetically modified organism with a lipid-forming pathway that is overexpressed or modified. In these or other embodiments, at least one of the microorganisms is a genetically modified organism with a lipid-forming pathway that is deleted.

In some embodiments, at least one of the microorganisms is a genetically modified organism with a sugar-consumption pathway that is modified or deleted so that the genetically modified organism only consumes acetic acid.

In various embodiments, the process further comprises recovering or generating a cellulose-rich material from the lignocellulosic biomass. For example, the cellulose-rich material may be selected from the group consisting of market pulp, dissolving pulp, fluff pulp, purified cellulose, functionalized cellulose, nanocellulose, paper, boxes, boards, pellets, and combinations thereof.

Compositions are provided as produced by the disclosed processes. For example, the present invention provides a lipid product. Apparatus may be configured for carrying out the processes disclosed, using known equipment (e.g., fermentors).

### DETAILED DESCRIPTION OF SOME EMBODIMENTS

This description will enable one skilled in the art to make and use the invention, and it describes several embodiments, adaptations, variations, alternatives, and uses of the invention. These and other embodiments, features, and advantages of the present invention will become more apparent to those skilled in the art when taken with reference to the following detailed description of the invention in conjunction with any accompanying drawings.

As used in this specification and the appended claims, the singular forms “a,” “an,” and “the” include plural referents unless the context clearly indicates otherwise. Unless defined otherwise, all technical and scientific terms used herein have the same meaning as is commonly understood by one of ordinary skill in the art to which this invention belongs. All composition numbers and ranges based on percentages are weight percentages, unless indicated otherwise. All ranges of numbers or conditions are meant to encompass any specific value contained within the range, rounded to any suitable decimal point.

Unless otherwise indicated, all numbers expressing parameters, reaction conditions, concentrations of components, and so forth used in the specification and claims are to be understood as being modified in all instances by the term “about.” Accordingly, unless indicated to the contrary, the numerical parameters set forth in the following specification and attached claims are approximations that may vary depending at least upon a specific analytical technique.

The term “comprising,” which is synonymous with “including,” “containing,” or “characterized by” is inclusive or open-ended and does not exclude additional, unrecited elements or method steps. “Comprising” is a term of art used in claim language which means that the named claim elements are essential, but other claim elements may be added and still form a construct within the scope of the claim.

As used herein, the phase “consisting of” excludes any element, step, or ingredient not specified in the claim. When the phrase “consists of” (or variations thereof) appears in a clause of the body of a claim, rather than immediately following the preamble, it limits only the element set forth in that clause; other elements are not excluded from the claim as a whole. As used herein, the phase “consisting essentially of” limits the scope of a claim to the specified elements or method steps, plus those that do not materially affect the basis and novel characteristic(s) of the claimed subject matter.

With respect to the terms “comprising,” “consisting of” and “consisting essentially of,” where one of these three terms is used herein, the presently disclosed and claimed subject matter may include the use of either of the other two terms. Thus in some embodiments not otherwise explicitly recited, any instance of “comprising” may be replaced by “consisting of” or, alternatively, by “consisting essentially of.”

All references in this disclosure to “acetic acid” are meant to also include acetate ions and acetate salts that may be formed or present. The amount of acetic acid that may be present in associated form, versus dissociated form or salt form, will depend on pH as well as the concentration of bases present.

The present invention, in some variations, is premised on the realization that an important aspect of commercial production of cellulosic sugars is the presence of potentially large amounts of acetic acid derived from the starting biomass. By first fermenting acetic acid to lipids or other products, and optionally removing the lipids or other products, the resulting fermentation of remaining sugars to desired products can be more efficient.

For example, conversion of acetic acid to lipids may be incorporated into Green Power+® processes as a way to remove acetic acid and convert it to something more useful. Also, conversion of acetic acid to lipids may be incorporated into AVAP® processes as a way to remove acetic acid and convert it to something more useful. The amount and concentration of acetic acid in solution will depend on the nature of the starting feedstock (hardwoods and agricultural residues generally having more acetyl content than softwoods), as well as extraction conditions employed.

In some variations, fermentation of acetic acid is carried out prior to fermentation of sugars. In some variations, a co-fermentation is employed wherein two microorganisms (e.g., two yeasts) or a single engineering microorganism (e.g., a genetically modified yeast) ferment acetic acid to lipids while simultaneously fermenting sugars to a desired fermentation product (e.g., ethanol).

When fermentation of acetic acid is carried out prior to fermentation of sugars, such fermentations may be conducted in the same fermentor or in different fermentors. When using different fermentors, each fermentor may be separately optimized for fermentation pH, temperature, dilution rate, aeration, additives, and other conditions. When using a single fermentor, such conditions may be adjusted during fermentation to first favor acetic acid consumption and then later favor sugar consumption, for example. For instance, it may be desirable to operate fermentation of acetic acid to lipids at a pH near 7, while fermentation of sugars to ethanol may be operated at a lower pH and without aeration.

In some variations, acetic acid is consumed and produces one or more products other than lipids. For example, acetic acid may be converted to carbon dioxide, cell mass, or other organic compounds. When acetic acid is consumed, its role as a fermentation inhibitor may be reduced, regardless of the final product(s) produced from the acetic acid. Therefore it will be recognized that the acetic acid fermentation need not necessarily be conducted at high rates or yields, but enough to avoid acetic acid inhibition for the sugar fermentation. For example, in some embodiments, acetic acid concentration is reduced from about 10-20 g/L down to about 5-10 g/L, such as about 8 g/L acetic acid.

In some embodiments, acetic acid is converted to lipids in a first fermentation, followed by fermentation of sugars to a product which acts as an extraction solvent for the lipids from the lipid-producing organism. This would be desirable to recover the lipids as a product, or a mixture of the two fermentation products (i.e. lipids dissolved in another product).

According to Chi et al., “Oleaginous yeast Cryptococcus curvatus culture with dark fermentation hydrogen production effluent as feedstock for microbial lipid production,” International Journal of Hydrogen Energy, 36 (2011), 9542-9550, which is hereby incorporated by reference herein, under the right pH and aeration conditions the consumption of organic acids of about 0.5 g/L/hr was observed. If the sugar consumption pathway is disrupted and aeration is provided to the fermentor, the acetic acid can be removed in under 30 hours. This prefermentation step could then be used to prepare the sugars for processing by anaerobic C₅-C₆ yeasts. The lipid-laden yeast may be recovered and recycled or disposed.

U.S. Patent App. Pub. No. 2013/0065282 to Tran et al. is also hereby incorporated by reference herein, for its teachings regarding microorganisms capable of converting acetic acid to lipids. This disclosure also incorporates by reference US 2008/0155000 and WO 2013/081456 herein in their entireties.

Without being limited by theory, in the growth phase, yeast can take acetic acid with oxygen consumption and make fatty alcohols (such as for growth of cell walls). In some embodiments, selectivity to fatty alcohols and lipids may be increased by deletion of some part of the sugar processing mechanism in the yeast. Increasing the rate of conversion of acetic acid may also be accomplished by additional engineering to the yeast.

Some embodiments are premised on the realization that a lipid-forming pathway may be disabled. In this case, acetic acid may be metabolized to carbon dioxide and cell mass, but not to lipids. Again, acetic acid concentration may be sufficiently reduced so that it does not act as an inhibitor in sugar fermentation.

Other embodiments are premised on the realization that a sugar-consumption pathway may be disabled in a microorganism (such as an ethanol-producing yeast) so the microorganism can only consume acetic acid. The resulting microorganism should grow nicely on acetic acid under aerobic conditions and leave all sugars behind.

Using recently disclosed acetic acid-consuming yeasts to make ethanol with an acetic acid-containing hydrolyzate will work best with the use of a fed-batch culture. The reason is the concentration of acetic acid can be kept below the critical concentration that stops the growth and/or fermentation ability of the yeast. This configuration can handle high-acetate hydrolyzates and eliminate acetate inhibition. It will also allow the yeast to turn on alcohol dehydrogenases which convert aldehydes (e.g., furfural and HMF) to the corresponding alcohol.

Fed-batch culture is, in the broadest sense, defined as an operational technique in biotechnological processes where one or more nutrients (substrates) are fed to the bioreactor during cultivation and in which the product(s) remain in the bioreactor until the end of the run. It is also known as semi-batch culture. In some cases, all the nutrients are fed into the bioreactor. The advantage of the fed-batch culture is that one can control concentration of fed-substrate in the culture liquid at arbitrarily desired levels.

This disclosure therefore provides a way to deal with acetic acid derived from biomass, for fermentation of cellulosic sugars to ethanol (or other products). Fed-batch culture fermentations are employed to utilize acetic acid-consuming yeasts to make ethanol from an acetic acid-containing hydrolysate. The concentration of acetic acid can be kept below the critical concentration that stops the growth and/or fermentation ability of the yeast. This configuration can handle high-acetate hydrolysates and eliminate acetate inhibition. It will also allow the yeast to turn on alcohol dehydrogenases which convert aldehydes (e.g., furfural and HMF) to the corresponding alcohol.

Certain exemplary embodiments of the invention will now be further described. These embodiments are not intended to limit the scope of the invention as claimed. The order of steps may be varied, some steps may be omitted, and/or other steps may be added. Reference herein to first step, second step, etc. is for illustration purposes only.

In some variations, the invention provides a process for producing ethanol from lignocellulosic biomass, the process comprising:

(a) extracting hemicelluloses and a first amount of acetic acid from a feedstock comprising lignocellulosic biomass;

(b) hydrolyzing the hemicelluloses, using an acid catalyst or enzymes, to generate hemicellulose monomers and a second amount of acetic acid;

(c) fermenting at least a portion of the acetic acid to lipids using a suitable lipid-producing microorganism, thereby reducing acetic acid concentration;

(d) optionally removing at least a portion of the lipids, during or following step (c);

(e) fermenting at least a portion of the hemicellulose monomers to ethanol using a suitable ethanol-producing microorganism; and

(f) recovering the ethanol.

In some variations, the invention provides a process for producing ethanol from lignocellulosic biomass, the process comprising:

(a) extracting hemicelluloses and a first amount of acetic acid from a feedstock comprising lignocellulosic biomass;

(b) hydrolyzing the hemicelluloses, using an acid catalyst or enzymes, to generate hemicellulose monomers and a second amount of acetic acid;

(c) in a single fermentor, fermenting at least a portion of the acetic acid to lipids using a suitable lipid-producing microorganism, thereby reducing acetic acid concentration, while also fermenting at least a portion of the hemicellulose monomers to ethanol using a suitable ethanol-producing microorganism that is different than the lipid-producing microorganism;

(d) optionally removing at least a portion of the lipids, during or following step (c); and

(e) recovering the ethanol.

In some variations, the invention provides a process for producing ethanol from lignocellulosic biomass, the process comprising:

(a) extracting hemicelluloses and a first amount of acetic acid from a feedstock comprising lignocellulosic biomass;

(b) hydrolyzing the hemicelluloses, using an acid catalyst or enzymes, to generate hemicellulose monomers and a second amount of acetic acid;

(c) in a single fermentor, fermenting at least a portion of the acetic acid to lipids using a suitable lipid-producing microorganism, thereby reducing acetic acid concentration, while also fermenting at least a portion of the hemicellulose monomers to ethanol using the lipid-producing microorganism that is also capable of producing ethanol from the hemicellulose monomers;

(d) optionally removing at least a portion of the lipids, during or following step (c); and

(e) recovering the ethanol.

In some variations, the invention provides a process for producing ethanol from lignocellulosic biomass, the process comprising:

(a) extracting hemicelluloses and a first amount of acetic acid from a feedstock comprising lignocellulosic biomass;

(b) hydrolyzing the hemicelluloses, using an acid catalyst or enzymes, to generate hemicellulose monomers and a second amount of acetic acid;

(c) fermenting at least a portion of the acetic acid to CO₂ and/or cell mass using a suitable acetic acid-consuming microorganism, thereby reducing acetic acid concentration;

(d) optionally removing at least a portion of the CO₂ and/or cell mass, during or following step (c);

(e) fermenting at least a portion of the hemicellulose monomers to ethanol using a suitable ethanol-producing microorganism; and

(f) recovering the ethanol.

In some variations, the invention provides a process for producing ethanol from lignocellulosic biomass, the process comprising:

(a) extracting hemicelluloses and a first amount of acetic acid from a feedstock comprising lignocellulosic biomass;

(b) hydrolyzing the hemicelluloses, using an acid catalyst or enzymes, to generate hemicellulose monomers and a second amount of acetic acid;

(c) in a single fermentor, fermenting at least a portion of the acetic acid to CO₂ and/or cell mass using a suitable acetic acid-consuming microorganism, thereby reducing acetic acid concentration, while also fermenting at least a portion of the hemicellulose monomers to ethanol using a suitable ethanol-producing microorganism that is different than the acetic acid-consuming microorganism;

(d) optionally removing at least a portion of the CO₂ and/or cell mass, during or following step (c); and

(e) recovering the ethanol.

In some variations, the invention provides a process for producing ethanol from lignocellulosic biomass, the process comprising:

(a) extracting hemicelluloses and a first amount of acetic acid from a feedstock comprising lignocellulosic biomass;

(b) hydrolyzing the hemicelluloses, using an acid catalyst or enzymes, to generate hemicellulose monomers and a second amount of acetic acid;

(c) in a single fermentor, fermenting at least a portion of the acetic acid to CO₂ and/or cell mass using a suitable acetic acid-consuming microorganism, thereby reducing acetic acid concentration, while also fermenting at least a portion of the hemicellulose monomers to ethanol using the acetic acid-consuming microorganism that is also capable of producing ethanol from the hemicellulose monomers;

(d) optionally removing at least a portion of the CO₂ and/or cell mass, during or following step (c); and

(e) recovering the ethanol.

In some variations, the invention provides a process for producing lipids from lignocellulosic biomass, the process comprising:

(a) extracting hemicelluloses and a first amount of acetic acid from a feedstock comprising lignocellulosic biomass;

(b) hydrolyzing the hemicelluloses, using an acid catalyst or enzymes, to generate hemicellulose monomers and a second amount of acetic acid;

(c) fermenting at least a portion of the acetic acid to lipids using a suitable lipid-producing microorganism, thereby reducing acetic acid concentration;

(d) optionally removing at least a portion of the lipids, during or following step (c);

(e) fermenting at least a portion of the hemicellulose monomers to a fermentation product using a suitable microorganism; and

(f) recovering the lipids.

In some variations, the invention provides a process for producing lipids from lignocellulosic biomass, the process comprising:

(a) extracting hemicelluloses and a first amount of acetic acid from a feedstock comprising lignocellulosic biomass;

(b) hydrolyzing the hemicelluloses, using an acid catalyst or enzymes, to generate hemicellulose monomers and a second amount of acetic acid;

(c) in a single fermentor, fermenting at least a portion of the acetic acid to lipids using a suitable lipid-producing microorganism, thereby reducing acetic acid concentration, while also fermenting at least a portion of the hemicellulose monomers to a fermentation product using a suitable microorganism that is different than the lipid-producing microorganism;

(d) optionally removing at least a portion of the lipids, during or following step (c); and

(e) recovering the lipids.

In some variations, the invention provides a process for producing lipids from lignocellulosic biomass, the process comprising:

(a) extracting hemicelluloses and a first amount of acetic acid from a feedstock comprising lignocellulosic biomass;

(b) hydrolyzing the hemicelluloses, using an acid catalyst or enzymes, to generate hemicellulose monomers and a second amount of acetic acid;

(c) in a single fermentor, fermenting at least a portion of the acetic acid to lipids using a suitable lipid-producing microorganism, thereby reducing acetic acid concentration, while also fermenting at least a portion of the hemicellulose monomers to a fermentation product using the lipid-producing microorganism that is also capable of producing the fermentation product from the hemicellulose monomers;

(d) optionally removing at least a portion of the lipids, during or following step (c); and

(e) recovering the lipids.

In some embodiments, the fermentation product is capable of extracting the lipids from the lipid-producing microorganism. Optionally, the process of the invention further comprises extracting the lipids from the lipid-producing microorganism using an extraction solvent comprising the fermentation product. For example, the fermentation product may be selected from the group consisting of C₄ or higher alcohols, C₄ or higher fatty acids, C₄ or higher alkanes, and combinations thereof. In some embodiments, the extraction solvent comprises ethanol. In some embodiments, the extraction solvent further comprises lignin or a lignin derivative.

In some embodiments, step (a) utilizes steam and/or hot water, optionally with an extraction catalyst. In some embodiments, step (a) utilizes an extraction catalyst, a solvent for lignin, and water. In some embodiments, step (b) utilizes a sulfur-containing acid catalyst. Step (b) may utilize an acid catalyst derived from step (a).

In some embodiments of the invention, at least one of the microorganisms is a genetically modified organism with a lipid-forming pathway that is overexpressed or modified. In these or other embodiments, at least one of the microorganisms is a genetically modified organism with a lipid-forming pathway that is deleted.

In some embodiments, at least one of the microorganisms is a genetically modified organism with a sugar-consumption pathway that is modified or deleted so that the genetically modified organism only consumes acetic acid.

In some embodiments of the invention, at least one of the microorganisms is a genetically modified organism with an acetic-consuming pathway that is overexpressed or modified.

Apparatus may be configured for carrying out the processes disclosed, using known equipment (e.g., fermentors). In some embodiments, an aerated fermentor for acetic acid conversion may utilize a high-shear mixer (rather than an agitator or sparger) to provide oxygen/air for aerobic fermentation.

In various embodiments, the process further comprises recovering or generating a cellulose-rich material from the lignocellulosic biomass. For example, the cellulose-rich material may be selected from the group consisting of market pulp, dissolving pulp, fluff pulp, purified cellulose, functionalized cellulose, nanocellulose, paper, boxes, boards, pellets, and combinations thereof.

Compositions are provided as produced by the disclosed processes. For example, the present invention provides a lipid product. Apparatus may be configured for carrying out the processes disclosed, using known equipment (e.g., fermentors). In some embodiments, an aerated fermentor for acetic acid conversion may utilize a high-shear mixer (rather than an agitator or sparger) to provide oxygen/air for aerobic fermentation.

The process further comprises, in preferred embodiments, removing lignin derived from the lignocellulosic biomass. The lignin may be removed during hemicellulose hydrolysis, or following such step. Lignin removal may be integrated with removal of ethanol or other C₆ fermentation product. In certain embodiments, lipids formed from acetic acid react with or physically bind with lignin, and a lipid-lignin mixture or compound may be recovered.

The biomass feedstock may be selected from hardwoods, softwoods, forest residues, industrial wastes, pulp and paper wastes, consumer wastes, or combinations thereof. Some embodiments utilize agricultural residues, which include lignocellulosic biomass associated with food crops, annual grasses, energy crops, or other annually renewable feedstocks. Exemplary agricultural residues include, but are not limited to, corn stover, corn fiber, wheat straw, sugarcane bagasse, sugarcane straw, rice straw, oat straw, barley straw, miscanthus, energy cane straw/residue, or combinations thereof.

As used herein, “lignocellulosic biomass” means any material containing cellulose and lignin. Lignocellulosic biomass may also contain hemicellulose. Mixtures of one or more types of biomass can be used. In some embodiments, the biomass feedstock comprises both a lignocellulosic component (such as one described above) in addition to a sucrose-containing component (e.g., sugarcane or energy cane) and/or a starch component (e.g., corn, wheat, rice, etc.).

Various moisture levels may be associated with the starting biomass. The biomass feedstock need not be, but may be, relatively dry. In general, the biomass is in the form of a particulate or chip, but particle size is not critical in this invention.

In any of these variations or embodiments, the process may further include recovering or generating a cellulose-rich material from the lignocellulosic biomass. The cellulose-rich material may be recovered following an initial fractionation of feedstock, for example. Alternatively, or additionally, the cellulose-rich material may remain in a process stream along with extracted hemicelluloses and may remain during oligomer hydrolysis and fermentation, in some embodiments. The cellulose-rich material may be selected from the group consisting of market pulp, dissolving pulp, fluff pulp, purified cellulose, functionalized cellulose, nanocellulose, paper, boxes, boards, pellets, and combinations thereof.

In some embodiments, the cellulose-rich solids are utilized as pulp for production of a material (such as nanocellulose), pellet, or consumer product. Alternatively, or additionally, the cellulose-rich solids may be combusted to produce energy. The cellulose-rich solids may also be enzymatically hydrolyzed to produce glucose.

Fermentable sugars are defined as hydrolysis products of cellulose, galactoglucomannan, glucomannan, arabinoglucuronoxylans, arabinogalactan, and glucuronoxylans into their respective short-chained oligomers and monomer products, i.e., glucose, mannose, galactose, xylose, and arabinose. The fermentable sugars may be recovered in purified form, as a sugar slurry or dry sugar solids, for example. Any known technique may be employed to recover a slurry of sugars or to dry the solution to produce dry sugar solids.

In some embodiments, the fermentable sugars are fermented to produce biochemicals or biofuels such as (but by no means limited to) ethanol, isopropanol, acetone, 1-butanol, isobutanol, lactic acid, succinic acid, or any other fermentation products. Some amount of the fermentation product may be a microorganism or enzymes, which may be recovered if desired.

Any stream generated by the disclosed processes may be partially or completed recovered, purified or further treated, analyzed (including on-line or off-line analysis), and/or marketed or sold.

Apparatus may be configured for carrying out the disclosed processes using chemical-engineering principles known in the art as well as principles disclosed in commonly owned patents and patent applications, cited above and incorporated by reference herein.

In this detailed description, reference has been made to multiple embodiments of the invention and non-limiting examples relating to how the invention can be understood and practiced. Other embodiments that do not provide all of the features and advantages set forth herein may be utilized, without departing from the spirit and scope of the present invention. This invention incorporates routine experimentation and optimization of the methods and systems described herein. Such modifications and variations are considered to be within the scope of the invention defined by the claims.

All publications, patents, and patent applications cited in this specification are herein incorporated by reference in their entirety as if each publication, patent, or patent application were specifically and individually put forth herein.

Where methods and steps described above indicate certain events occurring in certain order, those of ordinary skill in the art will recognize that the ordering of certain steps may be modified and that such modifications are in accordance with the variations of the invention. Additionally, certain of the steps may be performed concurrently in a parallel process when possible, as well as performed sequentially.

Therefore, to the extent there are variations of the invention, which are within the spirit of the disclosure or equivalent to the inventions found in the appended claims, it is the intent that this patent will cover those variations as well. The present invention shall only be limited by what is claimed.

What is claimed is:

1. A process for producing ethanol from lignocellulosic biomass, said process comprising:
(a) extracting hemicelluloses and a first amount of acetic acid from a feedstock comprising lignocellulosic biomass;
(b) hydrolyzing said hemicelluloses, using an acid catalyst or enzymes, to generate hemicellulose monomers and a second amount of acetic acid;
(c) fermenting at least a portion of said acetic acid using a suitable acetic acid-consuming microorganism, thereby reducing acetic acid concentration;
(d) fermenting at least a portion of said hemicellulose monomers to ethanol using a suitable ethanol-producing microorganism; and
(e) recovering said ethanol.

2. The process of claim 1, wherein at least a portion of said acetic acid is fermented to carbon dioxide and/or cell mass.

3. The process of claim 2, said process further comprising removing at least a portion of said carbon dioxide and/or cell mass.

4. The process of claim 1, wherein said acetic acid-consuming microorganism is a lipid-producing microorganism, and wherein at least a portion of said acetic acid is fermented to lipids.

5. The process of claim 4, wherein said acetic acid-consuming microorganism is a genetically modified organism with a lipid-forming pathway that is overexpressed or modified.

6. The process of claim 1, wherein said acetic acid-consuming microorganism is a genetically modified organism with a sugar-consumption pathway that is modified or deleted so that said genetically modified organism only consumes acetic acid.

7. A process for producing ethanol from lignocellulosic biomass, said process comprising:
(a) extracting hemicelluloses and a first amount of acetic acid from a feedstock comprising lignocellulosic biomass;
(b) hydrolyzing said hemicelluloses, using an acid catalyst or enzymes, to generate hemicellulose monomers and a second amount of acetic acid;
(c) in a single fermentor, fermenting at least a portion of said acetic acid using a suitable acetic acid-consuming microorganism, thereby reducing acetic acid concentration, while also fermenting at least a portion of said hemicellulose monomers to ethanol using a suitable ethanol-producing microorganism that is different than said acetic acid-consuming microorganism;
(d) optionally removing at least a portion of said lipids, during or following step (c); and
(e) recovering said ethanol.

8. The process of claim 7, wherein at least a portion of said acetic acid is fermented to carbon dioxide and/or cell mass.

9. The process of claim 8, said process further comprising removing at least a portion of said carbon dioxide and/or cell mass.

10. The process of claim 7, wherein said acetic acid-consuming microorganism is a lipid-producing microorganism, and wherein at least a portion of said acetic acid is fermented to lipids.

11. The process of claim 10, wherein said acetic acid-consuming microorganism is a genetically modified organism with a lipid-forming pathway that is overexpressed or modified.

12. The process of claim 7, wherein said acetic acid-consuming microorganism is a genetically modified organism with a sugar-consumption pathway that is modified or deleted so that said genetically modified organism only consumes acetic acid.

13. A process for producing lipids from lignocellulosic biomass, said process comprising:
(a) extracting hemicelluloses and a first amount of acetic acid from a feedstock comprising lignocellulosic biomass;
(b) hydrolyzing said hemicelluloses, using an acid catalyst or enzymes, to generate hemicellulose monomers and a second amount of acetic acid;
(c) fermenting at least a portion of said acetic acid to lipids using a suitable lipid-producing microorganism, thereby reducing acetic acid concentration;
(d) optionally removing at least a portion of said lipids, during or following step (c); and
(e) recovering said lipids.

14. The process of claim 13, said process further comprising fermenting at least a portion of said hemicellulose monomers to a fermentation product using a suitable microorganism that is different than said lipid-producing microorganism.

15. The process of claim 13, said process further comprising fermenting at least a portion of said hemicellulose monomers to a fermentation product using said lipid-producing microorganism that is also capable of producing said fermentation product from said hemicellulose monomers

16. The process of claim 13, wherein said acetic acid-consuming microorganism is a genetically modified organism with a lipid-forming pathway that is overexpressed or modified.

17. The process of claim 13, said process further comprising extracting said lipids from said lipid-producing microorganism using an extraction solvent comprising said fermentation product.

18. The process of claim 17, wherein said fermentation product is selected from the group consisting of C₄ or higher alcohols, C₄ or higher fatty acids, C₄ or higher alkanes, and combinations thereof.

19. The process of claim 18, wherein said extraction solvent further comprises ethanol.

20. The process of claim 18, wherein said extraction solvent further comprises lignin or a lignin derivative.
